(19) United States
(12) Patent Application Publication
(10) Pub. No.: US 2010/0021929 A1
(43) Pub. Date: Jan. 28, 2010

(54) DETECTION OF A BIOMARKER OF ABERRANT CELLS OF NEUROECTODERMAL ORIGIN IN A BODY FLUID

(75) Inventor: David V. Pow, Mount Crosby (AU)

Correspondence Address: EDWARDS ANGELL PALMER & DODGE LLP, P.O. BOX 55874, BOSTON, MA, 02205, US

(73) Assignee: Newcastle Innovation Limited, of Industry Development Centre, Callaghan NSW (AU)

(21) Appl. No.: 12/408,340

(22) Filed: Mar. 20, 2009

Related U.S. Application Data
(60) Provisional application No. 61/120,695, filed on Dec. 8, 2008.

(30) Foreign Application Priority Data
Mar. 22, 2008 (AU) 2008901400

(51) Int. Cl. G01N 33/53

(52) U.S. Cl. 435/7.1

(57) ABSTRACT

Assays and kits for detecting aberrant cells of neuroectodermal origin in a body fluid of an individual, comprising testing for expression of GLAST1b as a biomarker of the cells are disclosed. Intact GLAST1b and/or fragments thereof may be detected in the fluid. Alternatively, another analyte indicative of the expression of GLAST1b by the cells may be detected. The assay is particularly suitable for detecting expression of aberrant neuronal populations such as resulting from brain hypoxia. The fluid can be cerebrospinal fluid (CSF).

### CROSS-REFERENCE TO RELATED APPLICATIONS

This application claims the benefit under 35 U.S.C. §119 of Australian provisional application No. 2008901400, filed Mar. 22, 2008, and U.S. provisional application Ser. No. 61/120,695, filed Dec. 8, 2008, the entire contents of which are incorporated herein by reference.

### FIELD OF THE INVENTION

The invention provides methods for the detection of GLAST1b in a body fluid of an individual as a biomarker of aberrant cells of neuroectodermal origin. The methods have application, although not exclusively, in evaluating the extent of damaged, degenerating or dying neurons and/or glial cells as a result of injury, trauma or neurological diseases or conditions.

### BACKGROUND OF THE INVENTION

Brain hypoxia is a patho-physiological condition characterised by a decrease of oxygen supply to the brain. It is caused by reduced blood supply or blood in which there is low oxygen concentration. The lack of oxygen impairs several highly energy-dependent transport and scavenger systems in the brain. For example, the reuptake of glutamate, a major excitatory neurotransmitter, is reduced after hypoxia. Excess glutamate in the synaptic cleft causes additional neurons to depolarise, triggering an excitotoxic state which can damage or kill neurons.

Current available diagnostic tools for hypoxia-induced neuronal damage are serum biomarkers (astroglial protein S100 and neuron-specific enolase) and in vivo imaging by magnetic resonance tomography (MRT) or positron emission tomography (PET).

The disadvantage of serum biomarkers enolase and S100 is that they not suitable for quantifying the risk of further damage after ischaemic events in the human brain. Moreover, both markers indicate cell death only. Currently, there are no biomarkers available predictive of hypoxia induced cell damage.

The in vivo imaging techniques MRT and PET are useful for assessing neuronal damage. However, they are non-specific and both techniques currently cannot be used to selectively image neurons exposed to hypoxic conditions.

Glutamate homeostasis in the brain is achieved via the actions of multiple glutamate transporters. GLAST, also known as EAAT1, is one of the two most abundant glutamate transporters in the adult [1,2,3]. There is growing evidence for the existence of multiple splice variants of each of the main glutamate transporters, and proteins corresponding to at least three alternate splicings of EAAT2 have been identified [4-6] along with mRNA for others [7-10]. mRNA for two alternate splicings of GLAST where exons are skipped have been described. GLAST1a arises from the splicing out of exon 3 [11] and is expressed in glial cells [12]. mRNA for an exon-9 skipping form of GLAST has also been described in humans [13].

A previous report indicated that when mRNA coding for GLAST1b tagged with a fluorescent protein is expressed in HEK293 cells, the translated protein is localized in the endoplasmic reticulum and lacks glutamate transport activity [13].

### SUMMARY OF THE INVENTION

Broadly stated, the invention stems from the finding that GLAST1b can act as a biomarker of aberrant neuronal populations, particularly damaged or degenerating neurons, and neurons which are in the process of; or are at risk, of dying. The invention also stems from the observation that GLAST1b can be detected in body fluid and so may be used for diagnostic purposes. In at least some forms, the invention provides diagnostic assays for determining the presence, or extent of, GLAST1b expression by cells of neuroectodermal origin as a result of injury, trauma, neurological diseases or conditions, and other physiological conditions.

More particularly, in one aspect of the invention there is provided an assay for detecting aberrant cells of neuroectodermal origin in an individual, comprising testing a sample of a body fluid from the individual for expression of GLAST1b as a biomarker of the cells.

Assaying for the presence, or extent of, GLAST1b expression can involve determining whether the sample contains GLAST1b or fragments thereof, or other molecule indicative of GLAST1b expression.

Thus, in another aspect of the invention there is provided an assay for detecting aberrant cells of neuroectodermal origin in an individual, comprising:

obtaining a sample of a body fluid from the individual; and

determining whether the sample contains an analyte selected from the group consisting of GLAST1b and/or fragments thereof or other molecule indicative of GLAST1b expression, the presence of the analyte in the sample being indicative of the presence of said aberrant cells in tissue of the individual.

The determination of whether the sample contains the analyte can be achieved by any assay protocol deemed appropriate. Moreover, as will be understood, the sample can be subjected to one or more purification steps to provide a purified preparation, and the purified preparation assayed for the presence or absence of the analyte.

Typically, the detection of the analyte in an assay as described herein will comprise tagging GLAST1b and/or fragments thereof with an agent for providing a detectable signal, and detecting the signal. Usually, the agent will be labelled with a molecule for providing the signal, Thus, in one or more embodiments, the assay may further comprise:

(a) providing an agent for tagging GLAST1b and/or fragments thereof;

(b) permitting the agent to tag any GLAST1b and/or fragments thereof present in the sample; and

(c) detecting the presence or absence of GLAST1b and/or fragments thereof tagged by the agent.

Alternatively, the analyte can, for example, be an antibody or binding fragment thereof specific for GLAST1b, and a method embodied by the invention can comprise assaying for the antibody or binding fragment thereof.

The term “cells of neuroectodermal origin” wherever used in this specification is to be taken to encompass neurons and glial cells, including Muller cells of the retina. Typically, the aberrant cells will be neuron and/or glial cells, and most usually, neurons.

In at least some forms, assays embodied by the invention have application in evaluating the presence or extent of damage or injury to such cells in brain and other tissues, such as may arise as a result of ischaemia and/or hypoxia (e.g., due to stroke or the like). That is, the greater the level of the analyte detected by the assay, the greater the level of aberrant cells expressing GLAST1b and thereby, the greater the level of damage or injury to the tissue.

Likewise, in at least some forms, assays as described herein have application in evaluating the extent and/or progression of neurological disease and conditions. The evaluation of the extent and/or progression of damage, injury or neurological disease can involve comparison of the level of the detected analyte with a reference or control level.

When used in the context of the present invention, the term “GLAST1b” refers to the exon 9 skipping form of GLAST and includes all forms of GLAST1b that may be detected by virtue of the presence of amino acid sequence arising from the splice site between exons 8 and 10 of nucleic acid encoding the protein. This includes full-length and truncated forms of GLAST1b. The detection of forms of GLAST1b can, for example, be achieved through specific antibodies targeting this region of the protein.

The term “aberrant” wherever used in this specification in relation to cells of neuroectodermal origin encompasses cells departing from the normal phenotype and includes cells that are anomalous in appearance, that are metabolically stressed, degenerating or dying including as a result of neurological diseases and conditions, and cells that have been subject to trauma or injurious insults, such as hypoxia.

The term “tagging” is to be taken to encompass within its scope associating with GLAST1b and includes binding to the protein.

Advantageously, at least some forms of assay embodied by the invention may provide a relatively rapid and simple way of providing an indication of the presence or extent of damage to tissues comprising cells of neuroectodermal origin. This can facilitate the making of decisions regarding the administration of suitable treatment to an individual whom presents with stroke, ischaemia or the like, pending further medical evaluation of the individual. Moreover, the reliance on ultrasound scans, computed axial tomography (CAT) scans, positron emission tomography (PET), magnetic resonance imaging (MRI) and nuclear magnetic resonance (NMR) scanning methods to identify the presence and/or extent of brain and other neuronal damage may also be reduced thereby providing significant health cost savings. In addition, assays as described herein in one or more forms may provide a rapid, cost effective way of monitoring damaged or injured such tissue, or for example, progression of neurological or other diseases and conditions which result in damage and the like to cells of neuroectodermal origin.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like that has been included in this specification is solely for the purpose of providing a context for the present invention. It is not to be taken as an admission that any or all of these matters form part of the prior art base or were common general knowledge in the field relevant to the present invention as it existed anywhere before the priority date of this application.

The features and advantages of the invention will become further apparent from the following detailed description of non-limiting embodiments.

### BRIEF DESCRIPTION OF THE ACCOMPANYING DRAWINGS

FIG. 1: Dot blots probed with GLAST1b antibody. (A) Membranes dotted with conjugates of peptides 1-3 at positions 1-3 respectively. Biotinylated BSA was applied at position 4 as a positive control for the DAB reaction (B) Blots of peptide 1 and the similar peptide for the exon-9 skipping form of EAAC1 at positions 1 and 2 respectively. The GLAST1b antibody is highly selective for GLAST1b.

FIG. 2: Immunolabelling for GLAST1b in rat cortex (A), rat superior collicus (B), human cortex (C), cat cortex (D), monkey cortex (E) and rat cerebellum (F). Small subsets of neurons are strongly labelled in cortices and colliculi. In cerebellum, labeling was predominantly associated with Bergmann glia in the molecular cell layer (M), and some astrocytes in the granular layer (G). Scale bars A, F=25 μm; B-E='10 μm.

FIG. 3: Double Immunofluorescence labelling for GLAST1b in rat cortex, in conjunction with N-terminal (A,B) or C-terminal GLAST (D,E). In all cases GLAST1b labeling is evident in populations of neurons that also exhibit labeling for GLAST. In some cases (D) the GLAST1b/GLAST labeled neurons exhibit significant abnormalities suggesting that they are dead or dying cells. Scale bars A=50 μm, B,C,D=10 μm.

FIG. 4: Immunolabelling for GLAST1b in perfusion-fixed cortices of a control pig (A) or in pigs which exhibit histological damage to white matter (B), some cortical grey mater (C) or extensive grey matter damage (D). Even in extensively damaged animals (D) the dentate gyrus (arrow) is typically unlabelled. h, hippocampus, c, cortex. Scale bar, 1 cm.

FIG. 5: Sections from hypoxic pig cortex double immunofluorescence labeled for GLAST1b (A) and C-terminal GLAST (B) or for GLAST1b (C) and N-terminal GLAST (D). GLAST1b and C-terminal GLAST are evident in neurones A) in damaged regions of the cortex. N-terminal GLAST was evident in astrocytes (arrow, a), some of which also contained GLAST1b. Neurones did not label for N-terminal GLAST. Scale bars=10 μm.

FIG. 6: Immunolabelling of the thalamus (A) from a hypoxically-challenged pig brain and the CA1 region of hippocampus (B) using two additional GLAST 1b antibodies. Abundant neuronal labelling was evident. Scale bars 50 μm.

FIG. 7: D-aspartate uptake (dark staining) into glial puncta surrounding unlabelled somata of cortical pyramidal neurons (N) in a normal control brain. Scale bar, 10 μm.

FIG. 8: Hypoxic live brain slices showing (A) uptake of D-aspartate into an astrocyte soma (arrow) abutting a larger unlabelled neuronal soma (N). Conversely, (B) D-glutainate is accumulated into a subset of neurons (N) but not into adjacent astrocyte soma (arrow). Scale bar=10 μM.

FIG. 9: Immunolabelling of post mortem brain cortex from a human patient with Alzheimer's disease (AD) using an antibody against the exon boundary region of GLAST1b. Low magnification views (A) illustrate the presence of scattered labelled neurons (arrows). At higher magnification (B) labelled neurons (N) typically exhibit morphological characteristics (including a prominent apically-directed primary dendrite) suggestive of them being mostly pyramidal cells though some neurons (C) appear dysmorphic (N*). Small astrocyte-like cells (a) were also labelled GM, grey matter, WM, white matter. Scale bars, A, 50 μm, B,C, 30 μm.

FIG. 10: Western blot showing detection of GLAST1b in cerebrospinal fluid (CSF) from pig with induced brain hypoxia.

FIG. 11: Western blot reflecting correlation of GLAST1b expression with degree of hypoxic injury in pig CSF.

### DETAILED DESCRIPTION OF EMBODIMENTS OF THE INVENTION

It has been found by the inventor that GLAST1b is expressed by aberrant neuron cell bodies and their processes on the outer cell membrane in both “grey” and “white” brain matter, and in particular but not limited to, layers IV and V of the brain cortex. The inventor has also found that Glast1b is expressed by at least some glial elements, such as damaged Muller cells in the retina. Hence, whilst assays as described herein have application in detecting damaged and degenerating neurons and glial cells and in particular, evaluation of the presence, or extent of, damage to neurons and glial cells of the brain, the invention is not limited thereto. That is, assaying for Glast1b may also be used to detect other aberrant cells of neuroectodermal origin.

GLAST1b appears to exert a dominant negative influence on full-length GLAST function. In particular, the inventors demonstrate that GLAST1b is expressed by neurons whilst normally spliced GLAST is expressed by astrocytes. Moreover, their studies show that GLAST1b can be localized to the plasmamembranes of those neurons that express this protein, indicating that it may be a functional plasmalemmal glutamate transporter The finding that GLAST1b is expressed by aberrant neurons provides a means for evaluating the extent of neuronal damage or neurological disease. Damage to neurons can arise in various ways including from tissue injury and trauma, as well as ischaemia or hypoxia as a result of cardiovascular conditions including atheroma, atherosclerosis and stroke. Brain tissue in particular is highly sensitive to hypoxia. Brain hypoxia is a common ailment with serious medical consequences. The incidence of brain hypoxia during birth is 2 in 1,000 full-term human births. Hypoxia can also be induced by events such as reduced placental blood flow (intrapartum hypoxia), drowning, drug overdose, asphyxiation caused by inhalation of smoke, very low blood pressure, strangulation, cardiac arrest, carbon monoxide poisoning, high altitudes, choking, chronic snoring, compression of the trachea, complications of general anaesthesia, and diseases that paralyse the respiratory muscles.

Other conditions which can lead to neuronal damage, degeneration or death include neurological and neurodegenerative conditions, and conditions associated with diabetes mellitus including both Type I and Type 2 diabetes mellitus. Such neurological and neurodegenerative conditions include β-amyloid associated diseases, Alzheimer's disease (AD), Parkinson's disease, motor neurone disease, Huntington's disease, white body dimentias, Lewis body dimentias, neurological and neuroparalytic diseases and conditions amongst others.

In one or more embodiments, the detection of GLAST1b in the body fluid may be used as a diagnostic marker of the disease or condition (e.g., Alzheimer's Disease), and/or the extent or severity of the disease or condition. Likewise, an assay as described herein may be employed to monitor its progression and/or response of the disease or condition to treatment.

The body fluid utilised in an assay embodied by the invention can be any body fluid in which GLAST1b, fragments thereof or other analyte indicative of GLAST1b expression can be detected. For example, the body fluid may be selected from the group consisting of cerebrospinal fluid (CSF), blood (including blood serum and plasma, and fractions thereof) and urine. CSF will typically be used for evaluating brain tissue injury, trauma or degeneration, and can be collected by lumbar puncture from individuals in the conventionally known manner.

CSF is essentially an acellular fluid, but the inventors have found that free GLAST1b and fragments thereof can be detected in CSF. Moreover, damage or disruption of the blood-brain barrier due to head injuries, damage and other trauma may also result in an immune system response resulting in autoantibodies being generated against GLAST1b. Similarly, antibodies specific for GLAST1b may be present in the blood of individuals suffering from neurological conditions such as Alzheimers disease (AD) or other neurological conditions in which GLAST1b is expressed by effected neurons or other cells of neuroectodermal origin. As such, blood and more typically blood serum or plasma (or other blood fractions) may be assayed for the presence of such antibody and/or binding fragments thereof in accordance with one or more embodiments of the invention. Assaying for autoantibody specific for GLAST1b (and/or binding fragments thereof) can employ an enzyme linked immunosorbent assay (ELISA) or other appropriate detection system. For example, detection of the antibody can utilise a peptide bound to a solid support which has an amino acid sequence comprising or consisting of the splice slit between exons 8 and 10 of nucleic acid encoding GLAST, and involve assaying for binding of the antibody to the peptide. The bound antibody can for example be detected by a second labelled antibody as described further below.

The agent used to test for the expression of GLAST1b can be any agent that can provide an indication of the expression of the protein. Similarly, any suitable testing protocol can be used. The detection of GLAST1b can be by direct or indirect detection of expression of the protein. Conveniently, the expression of GLAST1b can be assayed for in an in vitro assay detection protocol.

Antibodies offer a particularly suitable means for specifically tagging GLAST1b, fragments thereof or other analyte indicative of GLAST1b expression. The antibody can be a polyclonal antibody or mononclonal antibody specific for the protein although it is preferable that the antibody be a monoclonal antibody. The production of antibodies and monoclonal antibodies is well established in the art (e.g., see Antibodies, A Laboratory Manual. Harlow & lane Eds. Cold Spring Harbour Press, 1988, and any updates thereof). For polyclonal antibodies, a mammal such as a sheep or rat can be immunized with an antigenic fragment of GLAST1b expressed externally of the outer cell membrane of neurons, and anti-sera is then isolated from the mammal prior to purification of the antibodies generated against the GLAST1b antigen by standard affinity chromatography techniques such as Sepharose-Protein A chromatography. The immunized animal can be periodically challenged with the GLAST1b antigen to establish and/or maintain high antibody titer. To produce monoclonal antibodies, B lymphocytes can isolated from the immunized mammal and fused with immortalizing cells (e.g., myeloma cells) using somatic cell fusion techniques (eg., employing polyethylene glycol) to produce hybridoma cells (e.g., see Handbook of Experimental Immunology, Weir et al Eds. Blackwell Scientific Publications. 4th Ed. 1986). Selection of hybrid cells can be achieved by culturing cells in hypoxanthine-aminopterin-thymidine (HAT) medium, and hybridoma cells then screened for production of antibodies specific for the GLAST1b antigen by enzyme linked immunosorbant assay (ELISA) or other immunoassay.

Rather than intact antibodies, binding fragments of antibodies may be used to tag GLAST1b. The term “binding fragment of an antibody” as used herein is to be taken to encompass any fragment of an antibody that binds to GLAST1b. The term expressly includes within its scope Fab and (Fab′)₂ fragments as can be obtained by papain or pepsin proteolytic cleavage respectively, and variable domains of antibodies (e.g., Fv fragments), that are capable of binding GLAST1b under the conditions of the assay employed.

Strategies for identifying proteinaceous binding agents suitable for use in methods of the present invention include large scale screening techniques. Phage display library protocols provide an efficient way of testing a vast number of potential peptide agents. Such libraries and their use are well known. International Patent Application No. PCT/US01/27702 (WO 02/20722), for example, discloses the use of phage display libraries to identify peptides for targeting cell types for the delivery of imaging and therapeutic agents to the target tissue.

Phage display libraries express random transgenic peptides or antibody variable domain(s) of known length on the surface of the selected bacteriophage. Each phage clone displays a distinct such peptide sequence. The peptide sequences are fused with major or minor coat proteins of the selected phage type and can be produced by inserting random oligonucleotides in DNA encoding the coat protein, transfecting the resulting construct into a suitable host bacterial strain, and generating phage particles upon superinfection of the bacterial strain with helper phage. In vivo administration of phage libraries to mice has also previously been employed to identify specific targeting peptides. Such in vivo selection systems involve administration of the phage library and recovery of bound phage from the target tissue or cell type (e.g., Pasqualini, R., and Ruoslahti, E., 1996).

Peptides which bind to GLAST1b can be identified by contacting neurons expressing the protein to identify phage clones in the library which bind GLAST1b. Unbound phage is washed away and the remaining bound phage is recovered. The pool of bound phage can be enriched by subjecting the bound phage to a number of such biopanning cycles, wherein the bound phage is collected and amplified utilising suitable host bacteria before being subjected to the next cycle. The sequence of the binding peptide of an isolated phage clone can then be identified by sequencing the relevant coat protein of the clone, and comparing that sequence with the known sequence for the native phage coat protein.

DNA encoding for the identified peptide can be used for expression of the peptide or be modified to provide other such agents for use in methods of the invention utilising recombinant techniques well known in the art. In particular, fusion proteins incorporating peptide sequences found to bind to GLAST1b for use in assays embodied by the invention can be provided, and the use of such fusion proteins in methods embodied by the invention is expressly encompassed. For instance, nucleic acid encoding a fusion protein can be provided by ligating the DNA encoding the binding peptide with DNA encoding peptides having a desired three dimensional conformation and/or amino acid sequence by employing blunt-ended termini and oligonucleotide linkers, digestion to provide staggered termini as appropriate, and ligation of cohesive ends. Alternatively, polymerase chain reaction protocols (PCR) can be utilised to generate amplicons with complementary termini which can be ligated together.

In particular, peptides specific for GLAST1b can be fused or conjugated with a carrier protein or scaffold amino acid sequence which presents the agent for binding or maintains the peptide in a three-dimensional conformation required for binding with GLAST1b, or which enhances the affinity and/or avidity of the binding with GLAST1b. In addition, inversion of amino acids within a sequence may be undertaken to increase stability or inhibit enzymatic degradation to increase half life of the agent in vivo. Similarly, peptides which contain D rather than L amino acids and are they are thereby resistant to proteolytic cleavage, particularly by endopeptidases are specifically encompassed. Peptides and fusion proteins suitable for use in one or more methods of the invention can be synthesised or be expressed in vitro and purified from cell culture media using known techniques for administration to a mammal.

However, any suitable agent capable of tagging GLAST1b can be used in assays embodied by the invention. For instance, rather than peptides, labelled glutamate analogues may be employed. Particularly suitable analogues will preferentially or selectively bind to, or associate with, GLAST1b compared to other forms of GLAST, or other glutamate transporters or receptors. D-glutamate for instance is not a preferred substrate for classical glutamate transporters and so may find application in one or more assays as described herein.

The agent used for tagging GLAST1b can be labelled with any molecule which by its nature is capable of providing or causing the production of an analytically identifiable signal which allows the detection of binding or interaction of the agent with GLAST1b. Such detection may be qualitative or quantitative. The agent for tagging the protein can, for instance, be an imaging agent or radioisotope such as ³²P, ¹²⁵I, ¹³¹I, chromium-51 and cobalt-60 or a more short lived isotope such as ¹⁸F (eg., incorporated into fluoro-deoxy glucose (FDG)), technecium-99m, (Tc-99), strontium-82, rubidium-82, thallium-201 chloride, lutetium-177, yttrium-90, actinium-225, bismuth-213, dysprosium-165, holmium-166 and copper-64, an enzyme, a fluorescent label, a chemiluminescent molecule or an affinity label such as biotin, avidin, streptavidin and the like.

An enzyme can, for example, be conjugated with an antibody by means of coupling agents such as glutaraldehyde, carbodiimides, or for example, periodate although a wide variety of conjugation techniques exists. Commonly used enzymes include horseradish peroxidase, glucose oxidase, β-galactosidase and alkaline phosphatase amongst others. Substrates for enzyme based detection systems will generally be chosen for production a detectable colour change upon hydrolysis. However, fluorogenic substrates can also be used which yield a fluorescent product rather than a chromogen. Suitable fluorescent labels include fluorescein, phycoerythrin (PE) and rhodamine which emit light at a characteristic wavelength in the colour range following illumination with light at a different wavelength.

Any suitable assay protocol can be employed in an assay embodied by the invention can be employed including competitive and non-competitive assays. Suitable assays which can be used include radioimmunoassay, antibody capture and enzyme linked immunosorbent assays (ELISA). Such assays include those in which GLAST1b and/or fragments are detected by direct binding with a labelled antibody, and those in which the target antigen is bound by a first antibody, typically immobilised on a solid substrate (e.g., a microtitre tissue culture plate formed from a suitable plastics material such as polystyrene, agarose, sepharose and other commercially available supports such as beads formed from latex, polystyrene, polypropylene, dextran, glass or synthetic resins), and a labelled second antibody specific for the first antibody is used to form a GLAST1b and/or GLAST1b fragment-first antibody-second antibody complex that is detected by a signal emitted by the label. Such sandwich techniques in which the antigen is immobilised by an antibody for presentation to a labelled second antibody specific for the antigen are well known. An antibody can be bound to a solid substrate covalently utilising commonly used amide or ester linkers, or by adsorption, Protein detection techniques such as conventionally known staining techniques following agarose or polyacrylamide gel electrophoresis such as native or SDS-PAGE (e.g., silver or Coomassie blue staining), and Western blotting detection techniques can also be employed. In this instance, the level of tagged GLAST1b and/or fragments thereof can for example be evaluated by densitometry or other suitable qualitative or quantitative method.

Assay methodologies useful in embodiments of the invention and methods for labelling antibodies and peptides can be found in, for example, Current Protocols in Molecular Biology. Ausubel F M., John Wiley & Sons Inc. Enzyme based assay protocols are also described for instance in Handbook of Experimental Immunology, Weir et al., Vol. 1-4, Blackwell Scientific Publications 4^{th} Edition, 1986 and subsequent editions thereof.

Rather than full length GLAST1b, an antigenic fragment of GLAST1b which is exposed to the exterior of neurons on expression of the protein, or for example, a mutant form of GLAST1b can be used in an assay embodied by the invention. The mutant form can, for instance, be a truncated form of GLAST1b or modified form of the protein with one or more amino acid changes compared to wild-type, GLAST1b.

The level of GLAST1b and/or fragment(s) thereof detected in a sample can be compared to reference or control data to determine or evaluate the extent of GLAST1b expression by tissue (e.g., brain tissue) of the individual from whom the sample was obtained. The reference data can for example, be data obtained from individuals with various levels of established expression of GLAST1b or damaged or injured tissue, providing a range of levels indicative of increasingly extensive damage, injury or the like. Alternatively, the reference or control data may simply provide a discrete level above which indicates that the individual has suffered damage to neuronal or other tissue comprising cells of neuroectodermal origin.

Moreover, for example, the result from an assay of the invention can be a colour obtained by enzymatic cleavage of a substrate as described above, and the reference data can consist of a chart or guide against which the result is visually compared to obtain an indication of the presence or extent of the expression of GLAST1b and/or fragments of the protein.

The individual from which the sample to be assayed in accordance with embodiments of the invention can, for instance, be a member of the bovine, porcine, ovine or equine families, a laboratory test animal such as a mouse, rabbit, guinea pig, a cat, dog, a primate or human being.

The invention also expressly extends to the provision of a kit for use in an assay embodied by the invention. The kit may, for example, include one or more of an antibody, peptide or other agent for tagging GLAST1b, and reagent(s)s such as washing solutions, dilution buffers and the like together with instructions for use. The antibody or other molecule of the invention can be labelled and/or bound to a solid support. Particularly preferred kits are those provided for use in an RIA, ELISA or other type of immunoassay.

Optimal concentrations of agents for tagging GLAST1b and/or fragments thereof, temperatures, incubation times and other conditions for tagging GLAST1b as described herein can be readily determined by conventional assay methodology.

The invention will now be further described by reference to non-limiting Examples.

### Example 1

### Detection of GLAST1b in Tissue

Antibodies were raised against a unique amino acid synthetic peptide corresponding to the amino acids encoded by the splice site between exons 8 and 10 of GLAST to enable selective detection of GLAST1b (the antibodies are available from Prof. David Pow, The University of Newcastle, Newcastle, NSW, Australia). The aim of the present study was to determine if GLAST1b was present in the CNS and if so, its cellular compartmentalization.

### 1. Methods

Animal experiments complied with the guidelines of the National Health and Medical Research Council (NHMRC, Australia). Antisera were generated in rabbit [14], using the unique 11 amino acid peptide H₂N-QIITIRDRLRT (SEQ ID No. 1) of GLAST1b (referred to hereafter as peptide 1), which spans the splice region between exons 8 and 10, (see Macnab L T and Pow D V, (2007) [24], the contents of which is incorporated herein in its entirety by cross-reference). The peptide was coupled to porcine thyroglobulin, (Sigma, Castle Hill, Australia).

### 1.1.1 Dot Blots

To verify that the antibodies recognised the new splice site, peptide 1 was coupled to bovine serum albumin (BSA) for use in dot blots as previously described [14]. To confirm the antisera did not recognise the full length form of GLAST, two additional peptides H₂N— GQIITISITATA (SEQ ID No. 2) and H₂N-AVDWFLDRLRT™ (SEQ ID No. 3) (peptides 2 and 3) representing peptide sequences at the exon 8-9 and 9-10 boundaries were similarly conjugated to bovine serum albumin (BSA). To verify that the antiserum did not recognise the homologous exon 9 splice site in the related glutamate transporter EAAC1 the peptide H₂N-QIITIRDRFRT (SEQ ID No. 4) representing the splice site region was also tested. Sera were tested by dot blotting [14] using peptides conjugated to BSA. 1 μL of each conjugate was applied to PVDF membranes (Biorad, Sydney, Australia) and probed with the primary antisera or pre-immune sera at dilutions of 1:500 to 1:20,000. Detection was revealed using a biotinylated anti rabbit secondary antibody and streptavidin-borseradish peroxidase complex (both from Amersham), with DAB as a chromogen. A BSA-biotin conjugate (40 ng) was also applied to each membrane as a positive control.

### 1.1.2 Western Blotting

Brains and retinas from adult Dark Agouti rats were collected after euthanasia (sodium pentobarbital 100 mg/kg IP). Western blotting employed standard methods [14]. Pre-absorption of antisera (50 μg of peptide 1 per ml of diluted antiserum) was used to confirm specificity of the antiserum. Conversely, pre-absorption with the other peptides tested by dot blotting was used to verify that staining persisted and was thus not attributable to either normal GLAST, nor to alternately spliced forms of EAAT2 or EAAT3.

Immunoprecipitation of proteins from brain was also performed using standard methods. Briefly, caprylic acid purified immunoglobulin fractions of antiserum against the amino terminal region of GLAST were coupled to Affigel 10 beads (Biorad) and used to immunoprecipitate proteins. Proteins isolated using the GLAST antibody were then analysed by Western blotting using the GLAST1b antiserum.

Membranes were blocked using 5% BSA in Tris buffered saline, then probed using the immune, pre-immune or preabsorbed antiserum at a range of dilutions (1:500-1:50,000). Binding of primary antibodies was detected using the same methods as for dot blots.

### 1.1.3 Immunohistochemistry

Adult Dark Agouti rats (n=5), cats (n=2, marmoset monkey (n=2) were euthanized by overdose of (sodium pentobarbital; 100 mg/Kg I.P.) and fixed by perfusion with 4% paraformaldehyde in 0.1 M sodium phosphate buffer. Tissues were dehydrated, embedded in paraffin wax and immunolabelled using standard immunoperoxidase or immunofluoresence techniques [2]. Additional sections of human superior temporal coltex were derived from a previous study [15]. Immunolabelling patterns for GLAST1b were compared with those obtained using guinea pig antibodies raised against the N or C terminal regions of GLAST that should recognise all forms of GLAST. Controls included use of pre-immune serum and pre-absorption of dilute immune serum with 50 μg of peptide 1 per ml of diluted antiserum.

### 1.2 Results

### 1.2.1 Dot Blotting

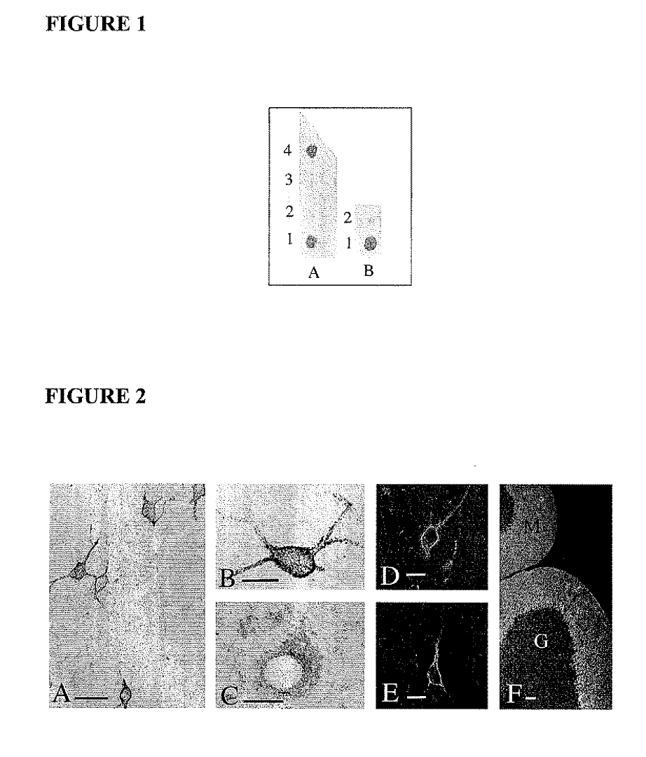

Initial screening by dot blotting demonstrated that the antiserum specifically recognised the peptide sequence that constituted the new splicing region formed by the skipping of exon 9, but did not recognise the original flanking peptides (FIG. 1A), nor the homologous sequence of the exon 9 skipping form of EAAC1 (FIG. 1B).

### 1.2.2 Western Blotting

Western blotting revealed a labelled band at around 50-55 kDa, which accords with the predicted molecular weight of GLAST1 b (data not shown). Pre-absorption of the antiserum resulted in no detectable labelling. Immunoprecipitation experiments confirmed the specificity of the GLAST1b antiserum used in this study, the GLAST1b antiserum detecting a protein band of around 50-55 kDa that had been immunoprecipitated by the GLAST antibody (data not shown).

### 1.2.3 Immunocytochemistry

Analysis of immunoperoxidase-labelled sagittal sections of rat brains revealed that GLAST1b was expressed by scattered populations of neurons, especially in layers IV and V of cortex in rats (FIG. 2A. Labelled neurons were also observed in inferior and superior colliculi (FIG. 2B). Sagittal sections of rat brain typically contained 5-20 labelled neuronal profiles, such cells often being present as small loosely associated clusters of 3-7 cells (FIG. 2A). Similar neurons were also detected in cortices of human (FIG. 2C). Immunofluorescence labelling of cat (FIG. 2D) and monkey (FIG. 2E) cortices also revealed labelled neurons. Labelling could also be discerned in many glial elements in the rodent brain, including the cerebellar Bergmann glia (FIG. 2F). In retina, GLAST1b was expressed by the Muller cells (data not shown).

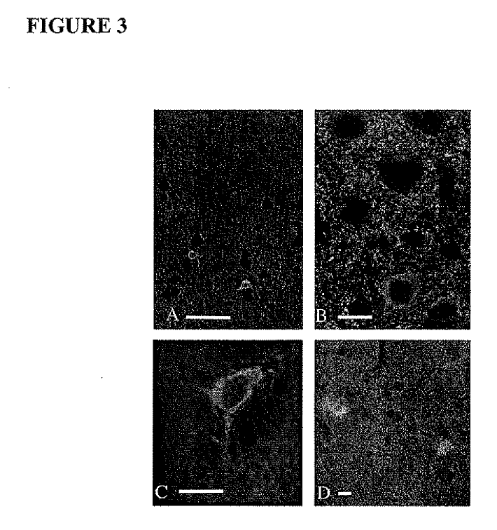

Double labelling for GLAST1b, and either the amino terminus of GLAST (FIGS. 3A, 3B) or the carboxyl terminus of GLAST (FIGS. 3C, 3D) was performed. Neurons that expressed GLAST1b exhibited immunolabelling for GLAST. Labelling for GLAST1b in neurons was punctate and apparently associated with plasmamembranes of the neurons. In some neurons that appeared to be degenerating (based on features such as blebbing of the plasmamembranes or an “exploded” appearance), labelling was evident in punctate intracellular inclusions (FIG. 3D). In all cases, labelling for normal GLAST was evident in cytoplasmic compartments of these neurons.

### 1.3 Discussion

The results show that GLAST1b protein is present in the nervous system. The finding that some neuons label for both GLAST and GLAST1b supports the view that GLAST was detected. However, labelling for GLAST extends throughout the soma of the labelled neurons, whereas GLAST1b labelling is restricted to plasmamembrane and some intracellular inclusions. This suggests that only some of the GLAST in the cell is GLAST1b, and that normally spliced GLAST may be co-expressed in the same neurons. Alternatively, the GLAST1b might under some circumstances by cryptic to detection by antibodies as has been demonstrated for other glutamate transporters such as GLT-1 and EAAT5 [15].

The localisation of GLAST1b to cortical and collicular neurons is in contrast to the primarily glial localisation of normally spliced GLAST and GLAST1a [12]. The incidence of GLAST1b-expressing neurons is relatively low. Immunolabelling for normal GLAST results in the staining of the glial sheaths surrounding neurons. Neuronal labelling can be successfully resolved by analysing thin sections (such as the paraffin wax sections used in this study).

The expression of GLAST1b in neurons accords with the prior observation that GLAST can be expressed in cortical neurons in Alzheimers disease [16]. The aberrant expression of alternate splicings of glutamate transporters in neurons has previously been reported in other disease states. For instance, splice variants of GLT-1 are expressed in neurons in disease states such as glaucoma [17] and in hypoxia [18]. In each case the conclusions drawn in these studies are that anomalies in local excitation induce the expression of glial glutamate transporters in the affected neurons as a protective mechanism.

Previous studies using tagged GLAST1b in vitro have suggested it would be targeted to intracellular locations. This does not appear to be an obligate state in the present study since GLAST1b was observed in plasmamembranes. Hence, GLAST1b may either function as a plasmalemmal glutamate transporter in such cells, or interact with other proteins such as full length GLAST or binding proteins such as NHERF1 [19], and thereby influence glutamate transport and homeostasis.

### 1.4 Conclusion

GLAST1b protein is expressed by populations of neurons in the brain which are anomalous in their morphology. The results of the present study show that GLAST1b expression can act as marker of aberrant neurons particularly populations that are about to die, possibly via excitotoxic mechanisms.

### Example 2

### Expression of GLAST1b in Pig Brain

The distribution of GLAST1b in the hypoxic neonatal pig brain was examined. In this model, the damage is variable between animals as assessed by independent blind scoring conducted by histological analysis on cresyl violet stained sections. Some animals typically experience only damage to white matter whilst others experience damage to either restricted regions of grey matter or in the most severe cases, to large areas of grey and white matter.

### 2.1 Methods

Animal experiments complied with the guidelines of the National Health & Medical Research Council (NHMRC) (Australia).

### 2.1.1 Animal Preparation

One day old pigs were anaesthetised using propofol (10 mg/kg/h) and alfentanil (50 μg/kg/h) iv. The pigs were intubated and ventilated using a neonatal ventilator, with oxygen and air to maintain arterial CO₂ at 35-45 mmHg and oxygen saturation 92-96%. A radiant warmer was used to maintain rectal temperature at 39.0±0.5° C. Following stabilisation of physiological variables for >20 minutes, hypercapnic hypoxia was induced by reducing FiO₂ to 10% and the ventilation rate to 10 bpm. FiO₂ was adjusted to maintain PaO₂ 15-20 mmHg for 45 minutes. The insult was terminated by returning the ventilation rate to 30 bpm and the FiO₂ to the lowest level necessary to maintain SaO₂ 95% or above. Pigs were then allowed to recover for 72 hours. This model results in variable damage to the brain as is the case with humans exposed to hypoxia [20]. Animals that died prematurely or did not suffer any detectable brain damage were excluded from this study. Brains from the eight remaining animals exposed to hypoxia and exhibiting subsequent brain damage were included in this study.

Control animals (n=6) were subjected to anaesthesia but no hypoxia then allowed to recover for 72 hours. Animals were euthanised by an overdose of sodium pentobarbital (120 mg/kg, I.P.). Brains were processed using two methods. Animals (N=3 control, N=3 hypoxic) were fixed by perfusion with 850 mL of 4% paraformaldehyde in 0.1M phosphate buffer, pH7.4, the brains were removed and sliced into 3 mm-thick slices using a slicing matrix and the slices were fixed by immersion in 500 mL of the same fixative for a further 3 hours. The remaining animal brains (N=3 control, N=5 hypoxic) were removed, sliced, and one half frozen for subsequent Western blotting analysis and the other half fixed for immunohistochemistry by immersion in 500 mL of 4% paraformaldehyde in 0.1M phosphate buffer, pH7.4 for 12 hours.

### 2.1.2 Antibodies

Antisera to GLAST1b were generated as described in Example 1. Two additional antisera against the same peptide were also generated. Other antisera used included antisera to the amino terminal and carboxyl terminal regions of GLAST, along with an antibody to GLT-1 which were previously generated and characterised [12].

An additional commercial monoclonal against glial fibrillary acidic protein (GFAP) was purchased from Sigma (Castle Hill, Australia), and a monoclonal antibody against microtubule associate protein 2 (clone MT01 Exbio) was purchased from Biocore, (Alexandria, Australia).

### 2.1.3 Western Blotting

Brains were rapidly collected after euthanasia. Western blotting employed standard methods [12,14]. Brain tissues (cortical sample encompassing cortical grey and white matter) were macerated under reducing conditions in ice-cold sample buffer (120 mM Tris, 4.8 mM EDTA, 0.024% SDS, 0.3M P3-mercaptoethanol, 10% glycerol) and a total protein sample created. Brain homogenates (10 μg of each sample) were subjected to 10% SDS-PAGE using a Mini-Protean 3 system (BioRad) and then transferred to PVDF membranes using a Mini Trans-Blot Cell (Biorad, Sydney, Australia). Transfers were routinely tested for efficiency by staining gels with Coomassie-blue (Sigma, Castle Hill, Australia) to verify that protein had been transferred out of the gels, whilst a second PVDF membrane was included to verify that “blow-through” of proteins through the first membrane did not occur. Molecular weight markers (Biorad) were run with all blots. Membranes were blocked using 0.5% skim milk powder in Tris-buffered saline, and then probed using each antiserum at a range of dilutions (1:1,000-1:50,000). Binding of the primary antibodies (directed against GLAST1b, or the carboxyl or amino terminal regions of GLAST) was detected using biotinylated secondary antibodies (Amersham, Castle Hill, NSW) at a dilution of 1:2,500, followed by streptavidin-biotin-HRP complex (Amersham) at a dilution of 1:2,500, with DAB as a chromogen. Pre-absorption of antisera (50 μg of peptide 1 per ml of diluted antiserum) was used to confirm specificity of the antiserum (data not shown).

### 2.1.4 Immunohistochemistry

Immunoperoxidase and immunofluorescence labelling was performed as previously described using standard methods [18]. Briefly, pig brains fixed with 4% paraformaldehyde in 0.1 M sodium phosphate buffer were then dehydrated through a graded series of water/ethanol solutions, cleared in xylene and embedded in paraffin wax [2]. Half-coronal sections of wax-embedded brains (8 μm in thickness) were cut on a rotary microtome and mounted onto silanated microscope slides. Sections were de-waxed with xylene and re-hydrated through a graded series of ethanol/water solutions and antigen recovery was performed using Revealit-Ag antigen recovery Solution (ImmunoSolution, NSW, Australia), For studies using DAB as a chromogen, sections were pre-treated with 3% hydrogen peroxide in methanol for 10 minutes (during the re-hydration process) to inhibit endogenous peroxidase activity. All sections were blocked in 0.5% bovine serum albumin (BSA)/0.05% Saponin/0.05% sodium azide in 0.1 M sodium phosphate buffer for 30 min before primary antibodies were applied. Secondary antibodies (biotinylated and fluorophore-coupled antibodies) and streptavidin-biotin horseradish peroxidase conjugates, all used at a dilution of 1:300, were purchased from Amersham (Castle Hill, Australia). Labelling for peroxidase-treated sections was revealed using DAB as a chromagen, and sections were mounted using DEPEX. Sections labelled using fluorophores were mounted in 50% glycerol in 0.1 M sodium phosphate buffer pH 7.2. Immunolabelling patterns for GLAST1b were compared with those obtained using antibodies raised against GLT-1a and with the patterns of labelling for the N or C-terminal regions of GLAST. An antibody against GLT-1b, which labels oligodendrocytes in the pig brain [18] was also used, to sensitively depict areas of white matter damage since GLT-1b labelling is readily lost in areas of white matter damage [unpublished data]. To clarify if GLAST1b immunoreactive cells were neurons or glia, or a mixture of both, additional double immunofluorescence labelling was performed using a mouse monolonal antibody against GFAP or a monoclonal antibody against MAP2. Labelling was revealed using species-specific secondary antibodies (Sigma, Castle Hill Australia) coupled to the fluorophores (Texas Red or FITC), each at a dilution of 1:300. Controls for labelling with the GLAST 1b, GLAST C-terminal an N-terminal antibodies included use of pre-immune serum and pre-absorption of dilute immune serum with 50 Hg of the immunising peptide per mL of diluted antiserum. Immunoperoxidase labelled sections were examined using an Olympus BX51 microscope equipped with an Olympus DP70 camera, whilst sections labelled using fluorophores were examined using a Nikon Cl confocal microscope.

### 2.1.5 Fluorojade Staining

Fluorojade staining was performed using Fluorojade C (Chemicon, Boronia, Australia) since this anionic fluorescent dye is thought to label degenerating neurons. Briefly, 8 μm thick brain sections were de-waxed and immunostained for GLAST1b as described above, using Texas Red as a fluorophore. Immunolabelled sections were then stained for 25 minutes with 0.0002% Fluorojade C in distilled water containing 0.1% acetic acid as per the manufacturers instructions. Sections were then rinsed with distilled water and mounted in 50% glycerol in PBS, and viewed immediately by confocal microscopy.

### 2.2 Results

### 2.2.1 Evoked Expression of GLAST1b Revealed by Immunocytochemistry

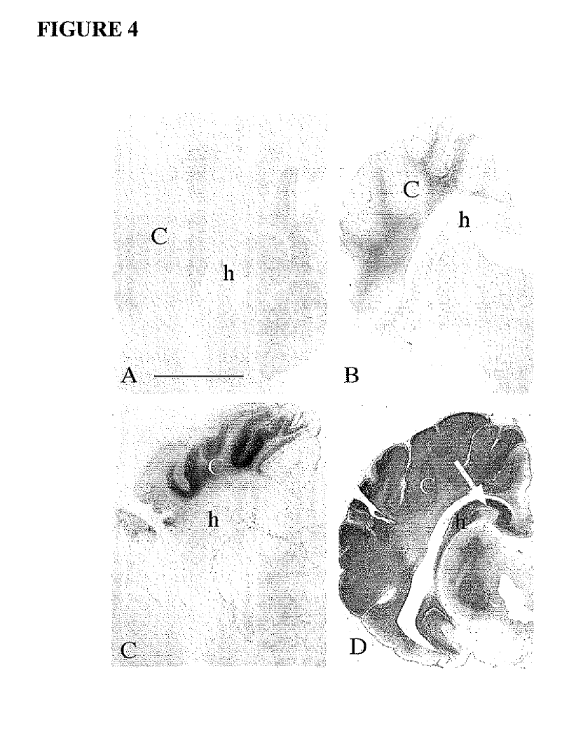

Analysis of immunoperoxidase-labelled coronal sections of control pig brains that had been fixed either by perfusion or immersion, revealed that in forebrain and midbrain regions there was very little, if any, expression of GLAST1b (FIG. 4A). Conversely, in brains subject to hypoxic insults there was an induction of expression of GLAST1b. In some brains where only white matter damage was evident, expression of GLAST1b was induced in white matter alone (FIG. 4B) whereas in others, induction was observed in restricted grey matter regions (FIG. 4C). Finally, in brains with large areas of cellular damage, GLAST1b was widely distributed, although even in these animals, some areas such as the dentate gyrus of the hippocampus, which are very resistant to damage, did not express GLAST1b (FIG. 4D). Additional brains fixed by immersion (due to the use of the contralateral side in Western blotting studies) showed similar patterns and intensities of immunolabelling.

The evoked expression of immunocytochemically-detectable GLAST1b was confirmed by Western blotting of samples from control brains or from brains that exhibited histological damage as previously described [18].

### 2.2.2 Western Blotting

Western blotting using the GLAST1b antibody in control pigs revealed a single band at ˜150-160 kDa which would accord with the molecular weight of a GLAST1b trimer complex as previously reported [1,3]. In hypoxic pigs that exhibited severe damage, the ˜150-160 kDa band was still present but was slightly diminished in intensity. Conversely, a strongly labelled band was evident around 50-55 kDa, which accords with the predicted molecular weight of monomeric GLAST1b. An additional prominent band was detected at ˜30 kDa. Since this was too small to represent full length GLAST1b, we hypothesised that this represented a cleavage product. A band at around 66-67 kDa was not observed with the GLAST1 b antibody indicating we did not detect normally spliced full length GLAST. Probing of Western blots with our C-terminal specific GLAST antibody also revealed a band of around 50-55 kDa in the hypoxic brains along with a similar ˜30 kDa band. As expected, this antibody also detected normal full length GLAST at ˜67 kDa. In contrast, our N-terminal specific GLAST antibody detected a broad band between 55 and 70 kDa but conspicuously did not detect either the 50-55kDa band or the ˜30kDa band. This suggested that the N-terminal antibody only detected full length GLAST and did not detect either GLAST1b or the GLAST1b fragment that we observe in this study. Pre-absorption of each antiserum resulted in no detectable labelling (data not shown).

2.2.3 GLAST1b is Expressed in Brain Regions that Lose Astroglial Expression of GLT-1a

In control pigs, GLT-1a was abundantly expressed in the forebrain. It was expressed by astrocytes in areas such as the hippocampus. The astrocytes exhibited immunolabelling for GLT-1a in all hippocampal layers whilst neurones were unlabelled. In contrast, there was little if any expression of GLAST1b in the control pig hippocampi. In such preparations, areas such as the CA1 exhibited a normal morphology as indicated by the presence in cresyl violet stained sections, of neurones with a plump and healthy appearance. However, in animals subject to hypoxia there was frequent loss of GLT-1a from the CA1 region of the hippocampus, and the neurones in such areas appeared to be abnormal, with a shrunken appearance as assessed by cresyl violet counterstaining of serial sections. Conversely, immunoreactive GLT-1a was normally retained in those astrocytes in the dentate gyrus region.

Analysis of serial sections revealed that in those brain regions where astrocytes lost their expression of GLT-1a, there was an induction of expression of GLAST1b, particularly in neurones. Thus in the hippocampus, GLAST1b was typically induced in the CA1 neurones. Such labelling was not restricted to the plasma membranes of the neurones, but was also present throughout the cell bodies and proximal dendrites of such cells. Conversely the astrocytes surrounding neurones in the dentate gyrus typically retained expression of GLT-1a and there was no evoked neuronal expression of GLAST1b in this region. Similar results were observed in other brain regions including cortex and thalamus (data not shown).

### 2.2.4 Double Labelling for GLAST1b and GFAP or MAP-2

To clarify whether the cells labelled for GLAST1b were neurons or glial cells or a mixture of both double-labelling for GFAP or MAP2 was performed. Some GLAST1b positive cells were found to double label for GFAP indicating they are likely to represent astrocytes. However, the majority of GLAST1b cells were immunoreactive for MAP2 suggesting that they were neurons.

2.2.5 Labelling for GLAST1b and Staining with Fluorojade

Staining for fluorojade and GLAST1b revealed that cells immunoreactive for GLAST1b were also stained with fluorojade.

2.2.6 Comparison of GLAST1b Expression with GLT-1a and N and C-Terminal GLAST

Examination of semi-serial sections (within 1-3 sections of each other, ie., separated by 24 microns at most) of areas such as the dentate gyrus revealed that where neuronal populations express GLAST1b. A similar neuronal expression of C-terminal region of GLAST is also observed. Conversely, analysis of N-terminal GLAST reveals no neuronal labelling in such regions. Instead the astrocytes around the GLAST1b immunoreactive neurones lack expression of immunocytochemically detectable N-terminal region of GLAST. This regional lack of astrocyte immunoreactivity for the N-terminal region GLAST was topographically comparable to the regional loss of GLT-1a in those astrocytes around GLAST1b immunoreactive neurones.

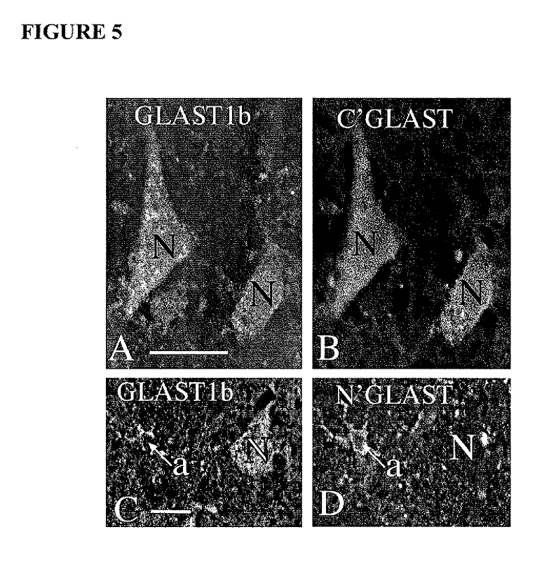

Double immunofluorescence labelling for GLAST1b (FIG. 5A) and the C-terminal region of GLAST (FIG. 5B) revealed that these two markers are co-localised to the same neuronal populations. Conversely double labelling for GLAST1b and N-terminal GLAST (FIGS. 5 C,D) revealed that GLAST1b immunoreactive neurones were not immunoreactive for N-terminal GLAST. This was not a methodological failure since occasional adjacent astrocytes that retained labelling for N-terminal GLAST were also labelled for GLAST1b.

2.2.7 Additional GLAST1b antisera confirm the evoked neuronal localisation of GLAST1b

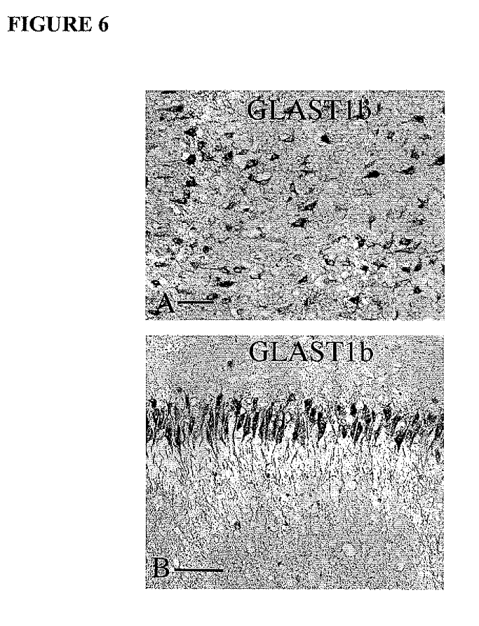

For confirmatory purposes the patterns of immunostaining using two additional antisera raised against GLAST1b were examined. Both antisera labelled populations of neurones in the hypoxic pig (FIGS. 6A,B).

### 2.2.8 White Matter Labelling

In some hypoxic brains, white matter damage was observed. Damage was initially identified in sections immunolabelled for GLT-1b as labelling for this oligodendroglial marker is lost in areas of white matter damage including areas of focal damage. This was confirmed by analysis of cresyl violet counterstained sections. In such GLT-1b deficient white matter areas, focal expression of GLAST1b was observed in sparse populations of cells. Higher magnification analysis of the same areas revealed cells with a variety of morphologies including neuronal-like morphologies and others with elongate cell bodies that may represent glial cells.

2.3. Discussion

The histochemistry results show that in response to hypoxia, there is a dramatically increased expression of GLAST1b in neurones in brain regions that are sensitive to damage and that such staining is coincident with staining for fluoro-jade staining which is often considered to be a marker for damaged cells. Some of the detected protein is present as high molecular weight species of around 160 kDa which was interpreted as GLAST1b trimers. This expression appears to be a sensitive marker of distressed neurones, since it is not induced in neurones in areas that are spared (such as the dentate gyrus neurones). That GLAST1b or a GLAST-like protein was detected is supported by the finding that immunoreactivity for the carboxyl terminal region of GLAST is also up-regulated in the same neurones. Conversely, the amino terminal region of GLAST is not detected in the neurones. This affirms that the GLAST protein detected is not the full length GLAST1b protein. This also accords with the finding that expression of the amino terminal-containing region of GLAST appears to be restricted to glial cells and moreover, that such glial GLAST is lost in areas of brain that are sensitive to damage by hypoxic insults.

In addition to the very prominent expression of GLAST1b in neurones, the identity of which was confirmed by double staining for the neuronal marker MAP-2, there is also a general rise in GLAST1b immunoreactivity in neuropil regions and white matter. The results further show that GFAP positive cells contribute to this staining, indicating that populations of astrocytes can also express GLAST1b.

Western blotting revealed, in homogenates of hypoxically insulted brains, an increased abundance of bands at ˜30 kDa and 50-55 kDa that were immunoreactive for both GLAST1b and the carboxyl terminal region of GLAST. It is believed that the 50-55 kDa band represents GLAST1b. However the lack of coincident labelling for the amino-terminal GLAST in neurones and the absence of comparable labelling of the 50-55 kDa band in Western blots evidences that the GLAST1b detected does not contain the normal amino terminal region of GLAST, or at least, does not contain immunoreactive epitopes for such. Similarly, it is believed the ˜30 kDa band represents a further truncated form of GLAST1b that retains the C-terminal region and exon 8-10 boundary regions but has lost the amino terminal half of the protein.

### 2.3.1 Intrinsic Expression of Multiple Forms or Fragments of GLAST in the Brain

At least one and possibly more alternate splicings or cleaved forms of GLAST are expressed even in the normal brain. A previous report [1] showed unambiguously that in brain regions such as cortex and olfactory bulbs, multiple bands representing slightly smaller forms of GLAST can be detected using a C-terminal directed antibody (A522). Similarly in a reconstituted system, it has been shown [21] this antibody detected a small (significantly less than 66 kDa) band that was immunoreactive for GLAST. The finding of a C-terminal epitope of GLAST at around 50-55 kDa using a C-terminal directed antibody is congruent with these findings.

The literature suggests that the vast majority of previous studies resolve a single band of around 65-67 kDa when using antibodies directed against the amino terminus of GLAST [eg., 22]. Only occasional studies have reported the detection of slightly smaller forms of GLAST when using antibodies against the amino terminal region [23]. These data suggest that amino terminal directed antibodies appear in most studies to predominantly detect full-length forms of GLAST rather than cleaved forms.

Minor modifications or alternate splicings of the amino terminal region are unlikely to account for the presence of the much smaller (˜30 kDa) band observed in the present study that is immunoreactive for C-terminal GLAST and GLAST1b. This cleavage product is likely to result from a sequence of modification events involving an initial cleavage of the extreme amino terminal region yielding the 50-55 kDa protein, followed by a subsequent cleavage to yield the ˜30 kDa fragment containing the exon 8-10 boundary and the C-terminal region.

### 2.3.2 Significance of GLAST1b Expression as a Marker of Neuronal Dysfunction in Hypoxia

In the present study, a profound up-regulation in expression of GLAST1b was demonstrated in those brain regions that are sensitive to hypoxic damage such as the CA 1 region of the hippocampus. This underscores the utility of GLAST1b or fragment(s) thereof in revealing the anatomical extent of damage in response to insults. Moreover, the expression of this protein at a very early stage after the insult, often before anatomical evidence of damage is easily discernable by histology, provides for a wider utility in a diagnostic context.

### Example 3

### D-glutamate is Accumulated by GLAST1b

A study was undertaken to evaluate accumulation of D-glutamate by GLAST1b. Briefly, hypercanic hypoxia was induced in one day old pigs essentially as described in Example 2.1.1. Control pigs were subjected to anaesthesia but no hypoxia and also allowed to recover for 72 hours as described above. The pigs were euthanased by an overdose of sodium pentobarbital, and the brains rapidly removed and placed into ice cold oxygenated artificial cerebrospinal fluid (CSF) (Ames media). 250 μm-thick slices were to room temperature before warming to 36° C., for the performance of transport studies. The temperatures used were slightly higher that those typically used for electrophysiology, and thus closer to physiological normality as transporter activity is greatly reduced if the temperature is significantly lowered. The neuroprotective effects of hypothermia that are evident at lower temperatures are also avoided since they are contraindicated in these studies.

D-aspartate (a substrate for classical glutamate transporters) or D-glutamate was added to the Ames media at a concentration of 20 μM and the slices permitted to actively accumulate the molecules for 75 minutes. Slices were then fixed with 2.5% glutaraldehyde in 0.1 M phosphate buffer for 12 hours. Specimens were washed with 0.1 M phosphate buffer, dehydrated with ethanol and embedded in epoxy resin according to standard methods previously applied to developing retinal tissues [22]. The uptake of D-aspartate or D-glutamate was revealed using specific antibodies raised against these synthetic molecules. Briefly, semi-thin (0.5 μm thick) sections were cut and immunolabelled using a rabbit polyclonal antiserum raised against D-glutamate antiserum [25] or D-aspartate antiserum [22] each at a dilution of1:10 000 as previously described [26].

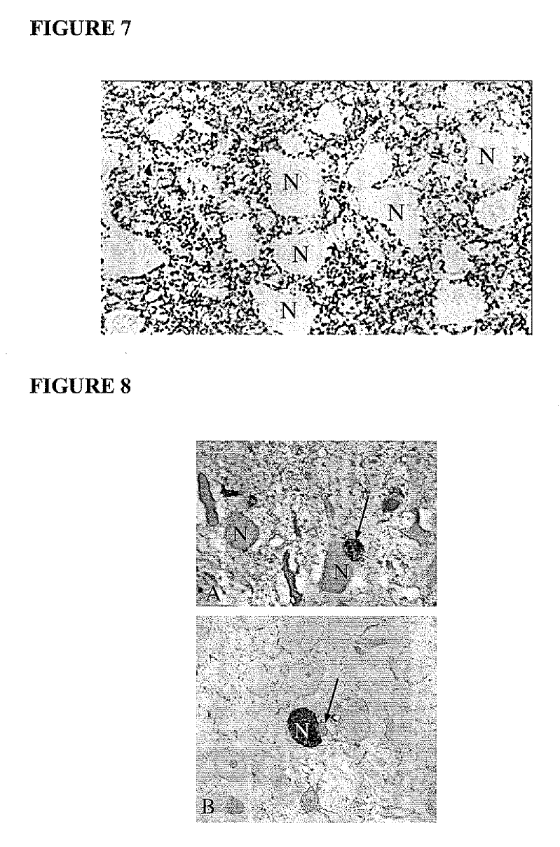

D-aspartate is a ligand for glial glutamate transporters and is normally accumulated into astrocytes but not neurons which therefore remain unlabelled. (see FIG. 7). D-glutamate is not normally a substrate for high affinity glutamate transporters and accumulation of this molecule is not observed into neurons in the normal brain. However, the uptake of D-glutamate is observed in hypoxic brains with expression of GLAST1b (see FIG. 8).

### Example 4

### Expression of GLAST1b in the Human Alzheimer Brain

### 4.1 Tissues

In this study, cortical samples (post-mortem human brain tissue) from control and Alzheimer patients were compared for expression of GLAST1b.

### 4.2 Immunohistochemistry

Immunolabelling was performed using standard protocols employing rabbit antibodies directed against the exon-9 skipping form of EAAT1 (GLAST1b), using biotinylated secondary antibodies (Amersham, Sydney, Australia) and streptavidin-biotin Horse radish peroxidase complex (Amersham, Sydney, Australia), labelling being revealed using diaminobenzidine as a chromogen. Appropriate controls such as pre-absorption of primary antisera with the immunising peptide and the use of pre-immune sera were also included. Each of these controls failed to yield positive staining (data not shown).

### 4.3 Results

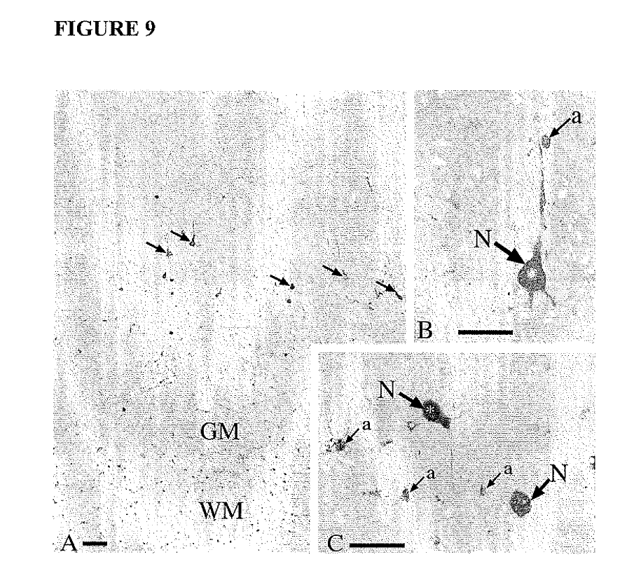

Use of the antibody against GLAST1b resulted in conspicuous labelling of populations of neurons. The neuronal labelling was diffuse, indicating the labelling multiple anatomical compartments including the plasma membranes (see FIG. 9).

### Example 5

### Detection of GLAST1b in Pig Cerebrospinal Fluid

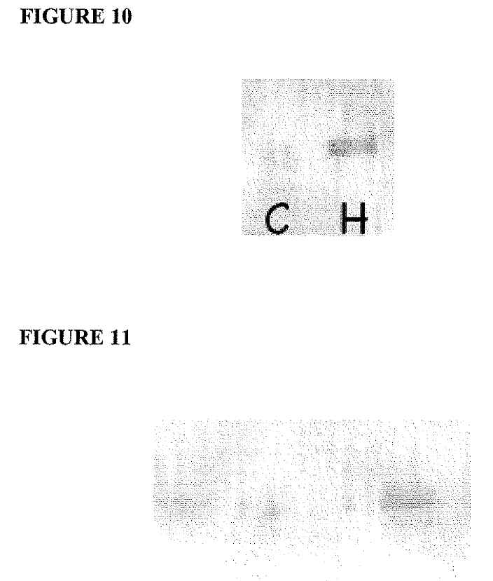

Cerebrospinal fluid (CSF) samples were obtained from pigs described in Example 3 at the point of euthanasia (sodium pentobarbital delivered IP) by lumbar puncture and prepared for Western blotting. Specifically, protein in the samples was denatured in a standard Western blotting sample preparation buffer containing sodium dodecyl sulfate (SDS) and mercaptoethanol as a reducing agent with heating to 85° C. for 10 mins. The prepared samples were then frozen until required. For the detection of GLAST1b, the samples were thawed, subjected to electrophoresis on 10% SDS PAGE gels, and protein was transferred to PVDF membranes by semidry transfer. The PVDF membranes were probed using a GLAST1b antibody, tagging being revealed using a biotinylated anti-rabbit secondary antibody followed by streptavidin-biotin-HRP complex. Diaminodenzidine was used as a chromogen. All of these methods are well known to the skilled addressee. As indicated by FIG. 10, GLAST1b was detected in CSF from pigs with induced hypercanic hypoxia (indicated by H) but not in control pig CSF (indicated by C).

In a further study, CSF samples were collected from control pigs and pigs subjected to different levels of brain hypoxia, and the samples assayed for GLAST1b by Western blot. The results are shown in FIG. 11 (lane 1 (left hand side) is control, lane 2 is CSF from a pig with histologically demonstrable brain injury, lane 3 is CSF from a pig subjected to hypoxia but with essentially no histological brain injury, and lane 4 is CSF from a pig with hypoxic brain injury. As can be seen, a distinct band is obtained from pig CSF when the pig has been subjected to hypoxia and suffers injury (lanes 2 and 4). The band is much weaker when the hypoxic insult essentially does not cause brain damage (lane 3), almost at control levels. The GLAST1b protein fragments detected in this study were approximately 25-35 kDa in size. The protein band detected is smaller than the intact GLAST1b protein, likely being indicative of cleavage fragments associated with the proteolysis of cells thus causing its release.

Although the invention has been described with reference to particular examples, it will be appreciated by those skilled in the art that numerous variations and/or modifications may be made without departing from the invention as broadly described. The present embodiments are, therefore, to be considered in all respects as illustrative and not restrictive.

### REFERENCES

1. Lehre K P, Levy L M, Ottersen O P, Storm-Mathisen J, Danbolt N C (1995) Differential expression of two glial glutamate transporters in the rat brain: quantitative and immunocytochemical observations. J Neurosci 3:1835-1853.
2. Williams S M, Sullivan N C, Scott H L, Finkelstein D I, Colditz P B, Lingwood B E, Dodd P R, Pow D V (2005) Glial glutamate transporter expression patterns in brains from multiple mammalian species. Glia 49:520-541.
3. Chaudhry F A, Lehre K P, van Lookeren Campagne M, Ottersen O P, Danbolt N C et al. Glutamate transporters in glial plasma membranes: highly differentiated localizations revealed by quantitative ultrastructural immunocytochemistry. Neuron. 1995; 15:711-720.
4. Reye P, Sullivan R, Scott H, Pow D V (2002c) Distribution of two splice variants of the glutamate transporter GLT-1 in the adult rat brain, Glia 38:246-255.
5. Sullivan R, Rauen T, Fischer F, Wiessner M, Grewer C, Bicho A et al. Cloning, transport properties, and differential localization of two splice variants of GLT-1 in the rat CNS: implications for CNS glutamate homeostasis. Glia. 2004; 45:155-169.
6. Rauen T, Wiessner M, Sullivan R, Lee A, Pow D V (2004) A new GLT-1 splice variant: cloning and immunolocalization of GLT1c in the mammalian retina and brain. Neurochem Int 45:1095-1106.
7. Utsunomiya-Tate N, Endou T, Kanai Y (1997) Tissue specific variants of glutamate transporter GLT-1. FEBS Lett 416:312-316.
8. Meyer T, Fromm A, Munch C, Schwalenstocker B, Fray A E, Ince P G, et al., The RNA of the glutamate transporter EAAT2 is variably spliced in amyotrophic lateral sclerosis and normal individuals. J Neurol Sci. 1999; 170:45-50.
9. Schmitt A, Asan E, Lesch K P, Kugler P (2002) A splice variant of glutamate transporter GLT1/EAAT2 expressed in neurons: cloning and localization in rat nervous system. Neuroscience 109.45-61.
10. Rozyczka J, Engele J. Multiple 5′-splice variants of the rat glutamate transporter-1. Brain Res Mol Brain Res. 2005 133:157-161.
11. Huggett J, Vaughan-Thomas A, Mason D. The open reading frame of the Na(+)-dependent glutamate transporter GLAST-1 is expressed in bone and a splice variant of this molecule is expressed in bone and brain. FEBS Lett. 2000; 485:13-18.
12. Macnab, L T, Williams S M, Pow D V (2006) Expression of the exon 3 skipping form of GLAST, GLAST1a, in brain and retina. Neuroreport 17:1867-1870.
13. Vallejo-Illarramendi A, Domercq M, Matute C. (2005) A novel alternative splicing form of excitatory amino acid transporter 1 is a negative regulator of glutamate uptake is a negative regulator of glutamate uptake. J. Neurochem. 95:341-348
14. Pow D V, Sullivan R, Scott H (2003) Antibody production and immunocytochemical localization of amino acid transporters. Methods Mol Biol 227:213-244.
15. Pow D V, Barnett N L. Developmental expression of excitatory amino acid transporter 5: a photoreceptor and bipolar cell glutamate transporter in rat retina. Neurosci Lett. 2000; 280: 21-24.
16. Scott, H., Pow, D. V., Tannenberg, A., and Dodd, P (2002) Aberrant expression of the glutamate transporter excitatory amino acid 1 (EAAT 1) in Aizheimer's disease. J. Neuroscience 22 RC206 (1-5)
17. Sullivan R K, Woldeinussie E, Macnab L, Ruiz G, Pow D V (2006) Evoked expression of the glutamate transporter GLT-1c in retinal ganglion cells in human glaucoma and in a rat model. Invest Opthalmol Vis Sci 47:3853-3859.
18. Pow D V, Naidoo T, Lingwood B E, Healy G N, Williams S M, Sullivan R K, O'Driscoll S, Colditz P B (2004) Loss of glial glutamate transporters and induction of neuronal expression of GLT-1B in the hypoxic neonatal pig brain, Brain Res Dev Brain Res153:1-11.
19. Lee A, Rayfield A, Hryciw D H, Ma T A, Wang D, Pow D, Broer S, Yun C, Poronnik P. Na(+)—H(+) exchanger regulatory factor 1 is a PDZ scaffold for the astroglial glutamate transporter GLAST. Glia. 2007; 55:119-129.
20. Björkman S T, Foster K A, O'Driscoll S M, Healy G N, Lingwood B E, Burke C, Colditz P B (2006) Hypoxic/Ischemic models in newborn piglet: comparison of constant FiO2 versus variable FiO2 delivery. Brain Res. 1100:110-117.
21. Haugeto O, Ullensvang K, Levy L M, Chaudhry F A, Honore T, Nielsen M, Lehre K P, Danbolt N C (1996). Brain glutamate transporter proteins form homomultimers. J Biol Chem 271:27715-27722.
22. Pow D V, Barnett N L (11999). Changing patterns of spatial buffering of glutamate in developing rat retinae are mediated by the Muller cell glutamate transporter GLAST. Cell Tissue Res. 297: 57-66.
23. Schlag B D, Vondrasek J R, Munir M, Kalandadze A, Zelenaia O A, Rothstein J D, Robinson M B (1998). Regulation of the glial Na+-dependent glutamate transporters by cyclic AMP analogs and neurons. Mol. Pharmacol. 53:355-369.
24. Macnab L T and Pow D V (2007). Central nervous system expression of the exon 9 skipping form of the glutamate transporter GLAST. Neuroreport 18:1867-1870.
25. Pow D V, Crook D K. 1996. Direct immunocytochemical evidence for the transfer of glutamine from glial cells to neurons: use of specific antibodies directed against the d-stereoisomers of glutamate and glutamine. Neuroscience. 70(1):295-302.
26. Pow D V, Crook D K. (1993) Extremely high titre polyclonal antisera against small neurotransmitter molecules: rapid production, characterisation and use in light- and electron-microscopic immunocytochemistry. J Neurosci Methods. 1993 June; 48(1-2):51-63.

1. An assay for detecting aberrant cells of neuroectodermal origin in an individual, comprising testing a sample of a body fluid from the individual for expression of GLAST1b as a biomarker of the cells.

2. An assay according to claim 1 wherein the testing for expression of GLAST1b comprises:
obtaining a sample of the body fluid from the individual; and
determining whether the sample contains an analyte selected from the group consisting of GLAST1b and/or fragments thereof, or other molecule indicative of GLAST1b expression, the presence of the analyte in the sample being indicative of the presence of the aberrant cells in tissue of the individual.

3. An assay according to claim 2 comprising determining whether the sample contains GLAST1b and/or fragments thereof.

4. An assay according to claim 2 wherein the analyte is an antibody specific for GLAST1b and/or binding fragments of the antibody, and the assay comprises assaying for the antibody and/or the binding fragments of the antibody.

5. An assay according to claim 1 wherein the cells are selected from the group consisting of neurons and glial cells.

6. An assay according to claim 5 wherein the cells are neurons.

7. An assay according to claim 1 for evaluating the extent of GLAST1b expression.

8. An assay according to claim 1 being an assay for diagnosing or evaluating neuronal or brain damage

9. An assay according to claim 8 being an assay for diagnosing or evaluating brain damage arising from brain trauma or injury.

10. An assay according to claim 9 wherein the damage is from hypoxia of the brain.

11. An assay according to claim 8 being an assay for evaluating neuronal damage arising from a neurological or neurodegenerative disease or condition.

12. An assay according to claim 11 wherein the neurological or degenerative disease or condition is Alzheimer's disease.

13. An assay according to claim 1 wherein the body fluid is cerebrospinial fluid.

14. An assay according to claim 1 wherein the individual is a human.

15. A kit for detecting aberrant cells of neuroectodermal origin in a body fluid from an individual, the kit including an agent for detecting expression of GLAST1b as a biomarker of the cells.


SEQUENCE LISTING (4 sequences)

SEQ ID NO: 1 - protein, 11 aa, Artificial Sequence
  Description of Artificial Sequence Synthetic peptide
QIITIRDRLRT

SEQ ID NO: 2 - protein, 12 aa, Artificial Sequence
  Description of Artificial Sequence Synthetic peptide
GQIITISITATA

SEQ ID NO: 3 - protein, 12 aa, Artificial Sequence
  Description of Artificial Sequence Synthetic peptide
AVDWFLDRLRTT

SEQ ID NO: 4 - protein, 11 aa, Artificial Sequence
  Description of Artificial Sequence Synthetic peptide
QIITIRDRFRT